(19) United States
(12) Patent Application Publication
(10) Pub. No.: US 2025/0387061 A1
(43) Pub. Date: Dec. 25, 2025

(54) PHYSICAL HUB FOR CABLES OF A PORTABLE MEDICAL DEVICE

(71) Applicant: Stryker Corporation, Portage, MI (US)

(72) Inventors: Gretchen Nicole Rudel, Woodinville, WA (US); Colette Louise Boileau, Redmond, WA (US); Neal Stanley Clark, Snohomish, WA (US); Tyler E. Marshall, Bothell, WA (US); Kristen Michelle Sebastianelli, Houston, TX (US); Lillian Aranka Steiner, Kirkland, WA (US); Rose Tingwei Yin, Bellevue, WA (US)

(73) Assignee: Stryker Corporation, Portage, MI (US)

(21) Appl. No.: 19/244,737

(22) Filed: Jun. 20, 2025

Related U.S. Application Data
(60) Provisional application No. 63/662,823, filed on Jun. 21, 2024.

(51) Int. Cl. A61B 5/273; A61B 5/00; A61B 5/1455; A61B 5/256; A61B 5/28

(52) U.S. Cl. CPC A61B 5/273; A61B 5/14552; A61B 5/256; A61B 5/28; A61B 5/6804; A61B 5/6892; A61B 5/742

(57) ABSTRACT

Systems, devices, and methods for connecting one or more sensors to a portable medical device using a hub are described herein. In some implementations, an example hub includes a device port configured to connect the hub to the portable medical device, and a first port and a second port configured to connect the hub to a first sensor and a second sensor, respectively. In some implementations, an example hub includes a first holder and a second holder configured to couple to a first cable and a second cable. The example hub is attached to a patient support apparatus or a garment.

### CROSS-REFERENCE TO RELATED APPLICATION

This application claims priority to U.S. Provisional Patent Application No. 63/662,823 filed on Jun. 21, 2024, which is incorporated herein by reference in its entirety as if fully set forth herein.

### BACKGROUND

Medical devices often require multiple wired connections. For example, a medical device, such as a monitor-defibrillator, may be connected to multiple sensors that are configured to monitor a patient experiencing a medical emergency. Each sensor may be placed at a different location on the patient, for instance. In some cases, the medical device is connected to another type of accessory device that enables the medical device to administer a treatment to the patient.

In particular emergency scenarios, any combination of the medical device, a sensor, an accessory device, or the patient may be repositioned while the medical device is in use. However, such repositioning can result in cables connecting the respective devices from being dislodged from respective ports, thereby interrupting the communicative connections between the devices. In some examples, a sensor or accessory device may malfunction while in use, and the sensor may need to be repositioned or replaced to continue monitoring and/or treatment of the patient. When the sensor or accessory device is physically disconnected from the medical device, patient care may be interrupted.

### BRIEF DESCRIPTION OF THE DRAWINGS

FIGS. 1A and 1B illustrate example environments in which a portable medical device is connected to a first sensor and a second sensor disposed on a subject.

FIG. 2 illustrates an example hub that includes contact sensors and locking mechanisms.

FIG. 3 illustrates example components of an example hub as described herein.

FIGS. 4A to 4C illustrate example hubs as described herein.

FIGS. 5A and 5B illustrates example processes for connecting sensors to a portable medical device.

FIG. 6 illustrates an example of an external defibrillator configured to perform various functions described herein.

### DETAILED DESCRIPTION

Implementations of the present disclosure are directed to specific improvements in the technical field of emergency medicine. In particular, implementations of the present disclosure relate to connecting multiple accessory devices to a portable medical device to monitor and/or treat a patient during a medical emergency.

Various implementations described herein relate to systems, devices, and methods for using a hub to connect accessory devices to a portable medical device. In various implementations, the hub connects to and/or routes cables that are connected between the accessory devices and the portable medical device. Various devices described herein can attach to a patient support apparatus and/or a garment. Accordingly, the cables can be secured closer to the patient to reduce tangling of the cables. In some examples, a cable can be secured closer to the position of the corresponding accessory device. Prompt identification of malfunctioning accessory devices and corresponding cables can improve use of sensors for monitoring and/or treatment of subjects experiencing a medical emergency. The use of various implementations described herein can improve the efficacy of medical treatments and reduce confusion for rescuers at the site of a medical emergency.

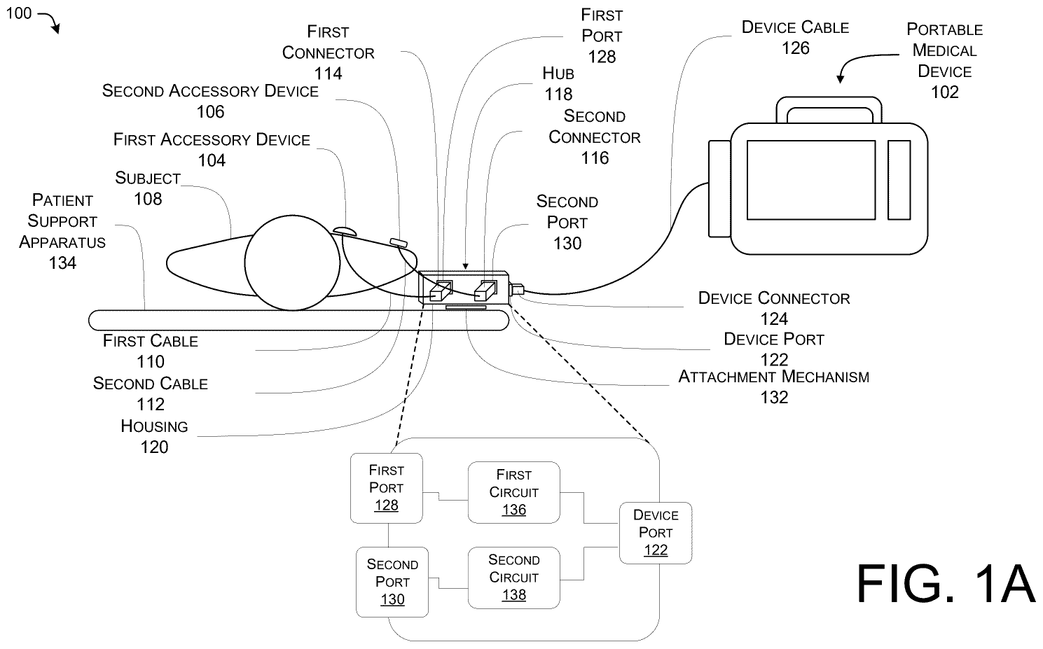

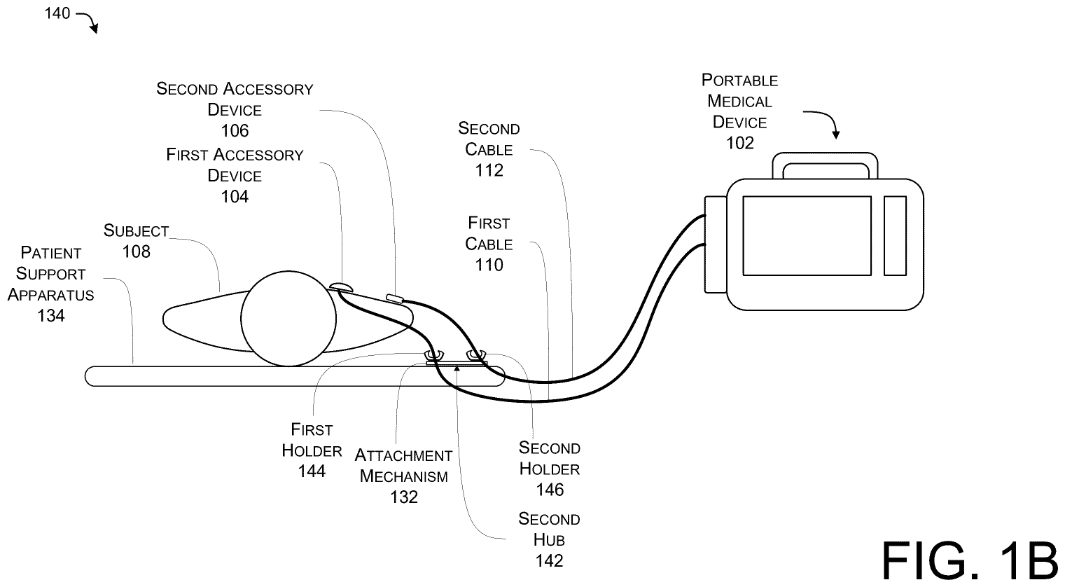

FIGS. 1A and 1B illustrates example environments 100 and 140 in which a portable medical device 102 is connected to a first accessory device 104 and a second accessory device 106 disposed on a subject 108. In various examples, the subject 108 is experiencing a medical emergency (e.g., cardiac arrest). The first accessory device 104 and the second accessory device 106 are configured to, in various examples, detect a physiological parameter of the subject 108 and/or deliver a treatment to the subject 108.

Starting with FIG. 1A, the portable medical device 102 is configured to monitor and/or treat the subject 108. In some examples, the portable medical device 102 is configured to be operated outside of a clinical environment by a rescuer. For instance, the environment could be at the scene of a car crash, in an airport terminal, in a residence, or some other non-clinical environment in which the subject 108 is experiencing a sudden medical emergency. In some cases, the rescuer brings the portable medical device 102 to the subject 108 in response to a report that the subject 108 has lost consciousness. In some examples, the rescuer is an emergency medical technician (EMT), a paramedic, a trained user, or the like. The portable medical device 102, for instance, may monitor and/or treat the subject 108 in the environment before the subject 108 is transferred to a clinical care environment, such as a hospital, for further care. The portable medical device 102 may be a monitor-defibrillator, a defibrillator, an ECG monitor, a vital sign monitor, an ultrasound machine, or another type of portable medical device.

In various examples, the first accessory device 104 and the second accessory device 106 are connected to the portable medical device 102. The first accessory device 104 and the second accessory device 106 are disposed on the subject 108. In various examples, the first accessory device 104 and the second accessory device 106 are configured to detect a physiological parameter of the subject 108 and/or deliver a treatment to the subject 108. The first accessory device 104 or the second accessory device 106 include, in various examples, an electrode, a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, a temperature sensor, or any other appropriate sensor. In some cases, the first accessory device 104 and the second accessory device 106 are configured to detect different types of ECGs (e.g., a single-, 3-, 5-, 6-, 12-, or 15-lead ECG, or another type of ECG). The first accessory device 104 and the second accessory device 106 may include different numbers of electrodes. For example, the first accessory device 104 is configured to detect a 3-lead ECG, and the second accessory device 106 is configured to detect a 5-, 6-, 12-, or 15-lead ECG. In some examples, the first accessory device 104 includes 10 electrodes, and the second accessory device 106 includes 3, 5, or 6 electrodes.

In some instances, the first accessory device 104 and the second accessory device 106 are electrically connected to a first cable 110 and a second cable 112, respectively. The first cable 110 and the second cable 112 are, in various cases, configured to provide, to the portable medical device 102, analog signals or data (e.g., digital signals) indicative of the analog signals detected by the first accessory device 104 and the second accessory device 106, respectively. The first cable 110 and the second cable 112 may be any suitable cable known in the art. In various examples, the first cable 110 and/or the second cable 112 may include a conductive material (e.g., copper, aluminum, or the like) configured to transmit electrical signals and/or an insulative material (e.g., polytetrafluoroethylene (PTFE), polyurethane (PU), silicone, polyethylene (PE), polyvinyl chloride (PVC), or the like) configured to shield the transmitted electrical signals from interference. The first cable 110 and the second cable 112, in some cases, may be a twisted pair cable, a coaxial cable, or another type of cable. In some instances, the first cable 110 and the second cable 112 may be selected based on durability, flexibility, shielding, conductivity, resistance, insulation, or any other characteristic that impacts performance. The first cable 110 and the second cable 112 are, in some examples, connected to a first connector 114 and a second connector 116, respectively. The first connector 114 and the second connector 116 may be configured to connect the first cable 110 and the second cable 112 to the portable medical device 102. Any cable described herein (including the first cable 110 and the second cable 112) may include at least one conductive wire that serves as at least one transmission interface. In some cases, a cable may include multiple conductive wires that respectively transmit different signals.

In various instances, it may be beneficial to reduce tangling and disorganization of the first cable 110 and the second cable 112. For example, during a time when the subject 108 is experiencing a medical emergency, the rescuer may connect the first accessory device 104 and the second accessory device 106 to the portable medical device 102. The first accessory device 104 and the second accessory device 106 may be disposed on the subject 108 to monitor and/or treat the subject 108. When the first accessory device 104 and the second accessory device 106 are in use, the first cable 110 and the second cable 112 may become tangled or disorganized, which may cause confusion for the rescuer and delay use of the accessory devices. In some examples, the cables may become tangled, and it may be difficult to determine which cable is associated with an accessory device. For instance, if a sensor is malfunctioning, it may be difficult to determine which sensor and/or cable corresponds to the malfunctioning sensor. If the cables are tangled, it may be difficult to identify and replace the malfunctioning sensor.

Moreover, there are other potential problems with connecting the first cable 110 and the second cable 112 directly to the portable medical device 102. In some examples, the portable medical device 102 and/or the subject 108 are moved during the medical emergency. For example, the portable medical device 102 may be moved to a location that is more convenient for a rescuer during the medical emergency. In some cases, the subject 108 is turned, lifted, carried, or otherwise repositioned in order to administer a treatment to the subject 108 (e.g., to position a mechanical chest compression device around the body of the subject 108), to improve the condition of the subject 108 (e.g., to clear an airway of the subject 108), or to transfer the subject 108 into a vehicle (e.g., an ambulance) for transport to a clinical environment. Due to the finite lengths of the first cable 110 and the second cable 112, the first cable 110 and/or the second cable 112 may unexpectedly become dislodged from the portable medical device 102 if the distance between the medical device 102 and the subject 108 changes during the movement.

These issues can be addressed, in some instances, by using a first hub 118 that is disposed adjacent to the subject 108 and configured to connect to the first cable 110 and the second cable 112. Accordingly, the use of the first hub 118 can enable use of shorter cables, avoid tangling of cables, and prevent the cables from being unexpectedly disconnected from the portable medical device 102 during movement. In some implementations, the first hub 118 may include ports configured to connect to first connector 114 and the second connector 116. The ports may be positioned adjacent to the location where a corresponding sensor is disposed on the subject 108. In various examples, the first hub 118 can provide an indication, to the rescuer, when the first cable 110 or the second cable 112 is unplugged or when the first accessory device 104 or the second accessory device 106 is malfunctioning. By providing an indication closer to the location of the unplugged or malfunctioning accessory device, the use of the first hub 118 can improve replacement and repositioning of the accessory device.

The first hub 118, in various implementations, is configured to electrically connect the first accessory device 104 and the second accessory device 106 to the portable medical device 102. The first hub 118, in various implementations, includes a housing 120. The housing may include any suitable material known in the art. In various examples, the housing includes a rigid material. In some examples, the housing may include a flexible material. For instance, the housing may include a material that can bend at a radius of curvature in a range of about 1 to 25 centimeters (cm). In some examples, the housing includes a material that can bend at a radius of curvature in a range of about 2 to 16 cm. In some instances, the housing includes a non-elastic material. For example, the housing may include a material with a Young's modulus greater than about 0.1 GPa. In some cases, the housing includes a material with a Young's modulus in a range of about 2 to 1500 GPa. In some instances, the housing includes a material with a Shore C hardness in a range of about 10 to 100. The housing, in some examples, includes a material suitable for healthcare settings. For example, the housing may include polycarbonate, acrylonitrile butadiene styrene, polypropylene, polyethylene, polyethylene terephthalate, ethylene vinyl acetate, polyurethane, a thermoplastic elastomer, natural rubber, neoprene, stainless steel, aluminum, carbon fiber, glass fiber, or another suitable material.

In various examples, a device port 122 is configured to connect the first hub 118 to the portable medical device 102. The device port 122, in some instances, is disposed in the housing. According to various implementations, the device port 122 is configured to couple to a device connector 124 of a device cable 126. The device cable 126 is configured to electrically connect to the portable medical device 102. The device cable 126 may be any suitable cable known in the art. In various instances, the device cable 126 includes an elastic cord, a stretchable cord, or a coil cord, or the like. The device cable 126, in some cases, includes a conductive material (e.g., copper, aluminum, or the like) and/or an insulative material (e.g., polytetrafluoroethylene (PTFE), polyurethane (PU), silicone, polyethylene (PE), polyvinyl chloride (PVC), or the like). The device cable 126, in some cases, may be a twisted pair cable, a coaxial cable, or another type of cable. According to various implementations, the device connector 124 includes a custom connector, a USB connector, a serial connector, or another suitable connector. In various examples, the device port 122 is a custom port, a USB port, a serial port, or another suitable port. In some implementations, the first hub 118 is connected to the portable medical device 102 by more than one device cable. For example, a separate device cable may be used for each accessory device, for each type of accessory device, for each type of cable port on the portable medical device 102, or for another reason.

In some instances, the device port 122 and the device connector 124 are configured to be coupled magnetically. For instance, the device port 122 and the device connector 124 may each include a magnetic material. In some examples, the device port 122 includes a magnet, such as a permanent rare earth magnet or an electromagnet, and the device connector 124 includes a ferromagnetic material. In some examples, the device port 122 includes the ferromagnetic material, and the device connector 124 includes the magnet. The ferromagnetic material may include steel, iron, nickel, cobalt, steel, or the like. The permanent rare earth magnet may include a neodymium alloy, a samarium alloy, a dysprosium alloy, or the like. The electromagnet, in various examples, includes a metal core (e.g., steel, iron, rare earth magnet, or the like) wrapped by a wire coil (e.g., copper wire, aluminum wire, or the like). Additional magnetic connectors and techniques for magnetic coupling are described, for example, in U.S. Pat. No. 9,306,322, which is incorporated herein by reference in its entirety as if fully set forth herein.

In some examples, the device port 122 and the device connector 124 are configured to be coupled mechanically. For instance, the device port 122 and the device connector 124 may be configured to couple using a snap-fit joint, a press-fit joint, a taper-fit joint, an interference-fit joint, a keyed-fit joint, a bayonet-fit joint, or the like. In various cases, the device port 122 and the device connector 124 may be configured to couple by a spring-loaded mechanism. Examples of the spring-loaded mechanism include, but are not limited to, a ball detent mechanism, a plunger latch mechanism, a slider latch mechanism, a toggle clamp, or the like.

In some instances, a first port 128 and a second port 130 are configured to connect the first accessory device 104 and the second accessory device 106, respectively, to the first hub 118. The first port 128 and the second port 130 are, in various cases, disposed in the housing. According to various implementations, the first port 128 is configured to couple to the first connector 114 of the first cable 110. In some implementations, the second port 130 is configured to couple to the second connector 116 of the second cable 112. The first connector 114 and the second connector 116, in various cases, include a custom connector, a USB connector, a serial connector, or another suitable connector. In some instances, the first port 128 and the second port 130 include a custom port, a USB port, a serial port, or another suitable port. In various examples, the first port 128 or the second port 130 may be configured to connect to multiple ECG sensors. For example, the first port 128 may be configured to connect to 12 electrodes. In some implementations, the first port 128 may be connected to the first accessory device 104 that is connected to 6 electrodes (e.g., a 6-lead ECG sensor). In various cases, the first port 128 may be connected to the first accessory device 104 and the second accessory device 106 that connected to 6 electrodes.

In various implementations, a first visual indicator and a second visual indicator are configured to indicate which port a connector is coupled to. For example, the first visual indicator may be disposed on the first port 128 and the first connector 114. The second visual indicator may be disposed on the second port 130 and the second connector 116. The first visual indicator and the second visual indicator may include a symbol, a color, or the like. In various cases, the first visual indicator and the second visual indicator include a light emitter configured to output a pattern of flashing (i.e., an LED turning on and off repeatedly) or a color of light. In some examples, the portable medical device 102 may provide an indication of which port a connector is coupled to. For instance, a visual display of the portable medical device 102 may display a diagram of each port and the corresponding accessory device. In various cases, the first hub 118 or the portable medical device 102 may provide an audio indication that a connector is connected to a port. The audio indication may include a beep, an audio tone, an audio alarm, a word, a phrase, or the like.

The first port 128 and the second port 130 may be disposed on the same side or on different sides of the housing. The first port 128 and/or the second port 130, in some cases, are disposed on the same side as the device port 122. In some examples, the first port 128 and/or the second port 130 are disposed on a different side than the device port 122.

In some examples, the first connector 114 and the second connector 116 are configured to couple magnetically to the first port 128 and the second port 130, respectively. For instance, the first port 128 and the second port 130 may include a magnet, such as a permanent rare earth magnet or an electromagnet, and the first connector 114 and the second connector 116 may include a ferromagnetic material. In various cases, the first port 128 and the second port 130 include the ferromagnetic material, and the first connector 114 and the second connector 116 include the magnet. In some examples, the first connector 114 and the second connector 116 are configured to couple mechanically to the first port 128 and the second port 130, respectively. For instance, the first connector 114 and the second connector 116 may be configured to couple to the first port 128 and the second port 130, respectively, by a snap-fit joint, a press-fit joint, a taper-fit joint, an interference-fit joint, a keyed-fit joint, a bayonet-fit joint, or the like.

In various implementations, a first locking mechanism is configured to prevent the uncoupling of the first port 128 and the first connector 114. A second locking mechanism is, in some examples, configured to prevent the uncoupling of the second port 130 and the second connector 116. The first locking mechanism and the second locking mechanism may be attached to the first port 128 and the second port 130, respectively, and/or the first connector 114 and the second connector 116, respectively. The first locking mechanism and the second locking mechanism include, in some cases, a magnetic connector, a twist-lock mechanism, a notch-lock mechanism, a latch, a screw-locking mechanism, a spring-loaded mechanism, or another suitable mechanism. Examples of a spring-loaded mechanism include, but are not limited to, a ball detent mechanism, a plunger latch mechanism, a slider latch mechanism, a toggle clamp, or the like.

The first hub 118 is configured to attach, via an attachment mechanism 132, to a patient support apparatus 134. In various examples, the patient support apparatus 134 includes a backboard, a stretcher, a gurney, a wheelchair, a mattress, a hospital bed, a cot, or the like. In some implementations, the first hub 118 is configured to attach to a surface of the patient support apparatus 134, a railing of the patient support apparatus 134, or a topper disposed on the patient support apparatus 134. The first hub 118, in some examples, is configured to attach to a garment of the subject 108 or a garment of the rescuer. The hub 118 may be configured to attach to a garment worn by the subject 108 or the rescuer, or a garment configured to be secured to the subject 108 or the rescuer (e.g., a vest, a strap, or the like). In various cases, the first hub 118 is configured to attach to a medical device (e.g., the portable medical device 102), medical equipment (e.g., a cart, an oxygen tank, a patient immobilization device, or the like), a vehicle, or another object at the scene of the medical emergency. The attachment mechanism 132, in various instances, includes a hook and loop fastener, a clip, a magnet, a snap fastener, a zipper, an adhesive, or any other suitable attachment mechanism 132. In various cases, the first hub 118 is configured to attach to one or more positions of the patient support apparatus 134, the garment, the medical device, the medical equipment, the vehicle, or the like. In various cases, the first hub is configured to attach to more than one of the patient support apparatus 134, the garment, the medical device, the medical equipment, the vehicle, or the like.

In various implementations, it may be beneficial to prevent uncoupling of the accessory devices from the subject 108. For instance, an object may catch on a cable (e.g., the device cable 126, the first cable 110, the second cable 112, etc.) and cause the first accessory device 104 to uncouple from the subject 108. In some cases, the subject 108, the first accessory device 104, the second accessory device 106, the hub 118, or the portable medical device 102 may be moved in order to improve monitoring or to administer a treatment to the subject. The first accessory device 104 or the second accessory device 106 may uncouple from the subject 108, causing an interruption in the monitoring and/or treatment of the subject 108.

These issues can be addressed, in various examples, by using an elastic component in the garment, the hub 118, or the attachment mechanism 132. In various examples, the garment may include an elastic material. For instance, if an object catches on the first cable 110, the garment may elastically deform to prevent the uncoupling of the first accessory device 104 from the subject 108. In some cases, the attachment mechanism 132 or the hub 118 include an elastic material or a spring. For example, the hub 118 may include a spring connected to the first port 128, second port 130, or the device port 122. In various instances, the subject 108 may be repositioned during the medical emergency, and a spring in the hub 118 may extend to prevent the uncoupling of the second accessory device 106 from the subject 108. The elastic material may include rubber, silicone, polyurethane, neoprene, ethylene propylene diene monomer (EPDM), latex, or any other elastic material. In various examples, the spring includes steel, titanium, a metal alloy, polyethylene, polypropylene, polyamide, polyether ether ketone, polyethylene terephthalate, or the like. A Young's modulus, in various examples, of the spring or elastic material may be in a range of 0.001 to 10. In some examples, the Young's modulus of the spring or elastic material is in a range of 0.005 to 0.5.

In some implementations, the first cable 110, the second cable 112, or the device cable 126 may be coupled to the garment or the patient support apparatus 134 to prevent uncoupling of the corresponding connector or port. In some examples, the first cable 110, the second cable 112, or the device cable 126 is coupled to the hub 118. The first cable 110, the second cable 112, or the device cable 126 may be coupled to the attachment mechanism 132. The attachment mechanism 132, in various cases, includes a clip configured to couple to the first cable 110 and prevent the uncoupling of the first connector 114 from the first port 128. In some instances, the cables can be coupled using a clip, a clamp, a latch, a notch, a spring-loaded mechanism, an adhesive, hook and loop fastener, a magnet, a snap fastener, or the like.

The first port 128 and the device port 122 are electrically connected by a first circuit 136, in some examples. The second port 130 and the device port 122 are electrically connected by a second circuit 138, in some cases. The first circuit 136 and the second circuit 138 are configured to provide analog signals or data indicative of the analog signals from the first port 128 and the second port 130, respectively, to the device port 122. In some examples, the first circuit 136 and the second circuit 138 are electrically isolated from each other. In various implementations, the first circuit 136 and the second circuit 138 are disposed within the housing The first circuit 136 and the second circuit 138, in various cases, include an analog circuit and/or a digital circuit. Although not specifically illustrated in FIG. 1A, the first circuit 136 may include the first port 128 and a first contact in the device port 122, and the second circuit 138 may include the second port 130 and a second contact in the device port 122, wherein the first contact is electrically isolated from the second contact.

In some implementations, a first contact sensor and a second contact sensor are configured to detect when the first connector 114 and the second connector 116, respectively, are uncoupled from the first port 128 and the second port 130, respectively. The first contact sensor and the second contact sensor may be configured to connect to the first circuit 136 and the second circuit 138, respectively. In some the first contact sensor and the second contact sensor are a part of the first circuit 136 and a part of the second circuit 138, respectively. The first contact sensor and the second contact sensor may include a mechanical sensor (e.g., a spring-based sensor, a leaf switch, a reed switch, or the like). In some examples, the first contact sensor and the second contact sensor are configured to detect a change in electrical signals. For instance, the first contact sensor and the second contact sensor may include a capacitive sensor, an impedance sensor, a current sensor, a voltage sensor, an inductive sensor, a Hall effect sensor, or the like. The first contact sensor and/or the second contact sensor, in various cases, include a switch. In some examples, the first contact sensor and the second contact sensor are configured to transmit a signal indicative of the first connector 114 and the second connector 116, respectively, being uncoupled from the hub 118. In various examples, the first contact sensor and the second contact sensor may be configured to transmit the signal to a transmitter, a light emitter, a speaker, a circuit, a switch, or the like. In various examples, the first contact sensor and the second contact sensor are configured to provide the signal to the portable medical device 102. For instance, in response to receiving the signal, the portable medical device 102 may display, to the rescuer, an alert on a visual display. In some examples, the portable medical device 102 may provide an audio alert.

A transmitter, in various implementations, is configured to transmit a first signal, to the portable medical device 102, indicating that the first connector 114 is uncoupled from the first port 128. In some examples, the transmitter is configured to transmit a second signal, to the portable medical device 102, indicating that the second connector 116 is uncoupled from the second port 130. The transmitter, in various examples, may be configured to transmit signals using one or more wireless networks. Examples of wireless networks include WI-FI®, cellular networks, wireless local area networks (WLANs), and BLUETOOTH®. In some instances, the signals are electromagnetic (EM) signals, radio waves, or the like. In some examples, the transmitter transmits radio waves to the portable medical device 102 via a cell tower. In some cases, the transmitter is connected to a wireless modem in the portable medical device 102, such as a modem for engaging in WI-FI®, WIGIG®, WIMAX®, BLUETOOTH®, or infrared communication. In some cases, the transmitter is connected to a component in the portable medical device 102 that enables use of a communication network, such as a network interface card (NIC), a network adapter, a local area network (LAN) adapter, or a physical, virtual, or logical address to connect to the various external devices and/or systems.

The transmitter may be connected to the first contact sensor and the second contact sensor, respectively, by a switch, a circuit, or the like. In various examples, a first transmitter and the first contact sensor are connected to the first circuit 136, and a second transmitter and the second contact sensor are connected to the second circuit 138. In some implementations, the first contact sensor provides a first analog contact signal to the first circuit 136, and the second contact sensor provides a second analog contact signal to the second circuit 138. The first circuit 136, in various examples, provides the first analog contact signal or a digital contact signal indicative of the first analog contact signal to the portable medical device 102. The second circuit 138 may provide the second analog contact signal or a second digital contact signal indicative of the second analog contact signal to the portable medical device 102. The portable medical device 102, in some examples, is configured to output an alert that the first connector 114 and/or the second connector 116 is uncoupled.

In various examples, a first light emitter and a second light emitter are configured to provide an indication when the first connector 114 and the second connector 116, respectively, are uncoupled from the first port 128 and the second port 130, respectively. The first light emitter and the second light emitter, in some cases, include a light emitting diode (LED), a fluorescent bulb, or the like. The indication may include a pattern of flashing or a color of light. For instance, an LED may emit a green light when the first connector 114 is coupled to the first port 128 and a red flashing light when the first connector 114 is uncoupled from the first port 128. The first light emitter and the second light emitter may be connected to the first contact sensor and the second contact sensor, respectively, by a switch, a circuit, or the like. In some implementations, the first contact sensor and the first light emitter are connected to the first circuit 136, and the second contact sensor and the second light emitter are connected to the second circuit 138.

In some implementations, the hub 118 includes a speaker configured to provide an indication when the first connector 114 and the second connector 116, respectively, are uncoupled from the first port 128 and the second port 130, respectively. The speaker may, in various cases, output a sound (e.g., a beep, an audio tone, an audio alarm, or the like).

As illustrated in FIG. 1B, in various implementations of the present disclosure, the first cable 110 and the second cable 112 are coupled to a second hub 142 that includes a first holder 144 and a second holder 146. The first cable 110 and the second cable 112, in some examples, are connected to the portable medical device 102. The first cable 110 and the second cable 112, in some instances, are connected to the first accessory device 104 and the second accessory device 106, respectively.

In some instances, the first holder 144 and the second holder 146 are configured to couple to the first cable 110 and the second cable 112. The first holder 144 and the second holder 146 may be configured to couple to cables of different sizes. For instance, the first holder 144 and the second holder 146 may include a hinge, a spring, a joint, a sliding mechanism, a telescoping mechanism, a flexible material, an elastic material, or another component configured to enable size adjustment. In various examples, a first static coefficient of friction between the first holder 144 and the first cable 110 and a second static coefficient of friction between the second holder 146 and the second cable 112 are in a range of about 0.3 to 1.2. In some instances, the first static coefficient of friction and the second static coefficient of friction are in a range of about 0.5 to 1. In some cases, the first holder 144 and the second holder 146 are configured to decrease or prevent movement of the first cable 110 and second cable 112, respectively, relative to the first holder 144 and second holder 146, respectively. The first holder 144 and the second holder 146 include, in various cases, a third locking mechanism and a fourth locking mechanism, respectively.

The third locking mechanism, in various cases, is configured to prevent uncoupling of the first cable 110 and the first holder 144. The fourth locking mechanism, in some examples, is configured to prevent uncoupling of the second cable 112 and the second holder 146. The third locking mechanism and the fourth locking mechanism may include a clip, a clamp, a latch, a notch, a spring-loaded mechanism, a snap link, or the like.

The first holder 144, in some examples, is configured to attach to the second holder 146. The first holder 144 and the second holder 146 may be removably attached by a hook and loop fastener, a clip, a magnet, a snap fastener, a zipper, an adhesive, or the like. In various implementations, the first holder 144 is permanently attached to the second holder 146. According to some implementations, the first holder 144 is adjacent to the second holder 146. For example, the first holder 144 is, in various cases, attached directly to the second holder 146. In some examples, the first holder 144 is not adjacent to the second holder 146. For instance, the first holder 144 and the second holder 146 may be attached to a holder support. In some implementations, the holder support enables the first holder 144 and the second holder 146 to be positioned closer to the position of the first accessory device 104 and the second accessory device 106, respectively, on the subject 108. The holder support, in various cases, may reduce or prevent the tangling or overlapping of the first cable 110 and the second cable 112.

According to some examples, the holder support includes polycarbonate, acrylonitrile butadiene styrene, polypropylene, polyethylene, polyethylene terephthalate, polyurethane, ethylene vinyl acetate, polyurethane, a thermoplastic elastomer, natural rubber, neoprene, stainless steel, aluminum, carbon fiber, glass fiber, polyester, silicone, or another suitable material. The first holder 144 and the second holder 146 may be removably attached to the holder support. For instance, the first holder 144 and the second holder 146 may be removably attached to the holder support by a hook and loop fastener, a clip, a magnet, a snap fastener, a zipper, an adhesive, or the like. In some cases, the first holder 144 and the second holder 146 are permanently attached to the holder support.

In some examples, an attachment mechanism 132 is configured to attach the first holder 144 and the second holder 146 to the patient support apparatus 134, a garment, a medical device, a medical equipment, a vehicle, or the like. The attachment mechanism 132 may include the holder support. The attachment mechanism 132, in various cases, is configured to attach the first holder 144 and the second holder 146 to a surface of the patient support apparatus 134, a railing of the patient support apparatus 134, or a topper disposed on the patient support apparatus 134.

In various implementations, it may be beneficial to clean the first holder 144 and the second holder 146 to maintain functionality, prevent transmitting an infection to the subject 108 or the rescuer, and comply with regulations for healthcare equipment and healthcare facilities. The first holder 144 and the second holder 146 may include a material that can be decontaminated. For instance, the material may be non-porous and/or smooth. In some examples, the first holder 144 and/or the second holder 146 may have a coating that provides water resistance, chemical resistance, heat resistance, or antimicrobial properties. For instance, the coating may include a polyurethane coating, a silicone coating, an epoxy coating, a vinyl ester coating, a vinyl coating, a fluoropolymer coating, a polyimide coating, a phenolic coating, a silver-based coating, a copper-based coating, a triclosan-based coating, a chitosan-based coating, a polyhexamethylene biguanide-based coating, a zinc-based coating, or another suitable coating.

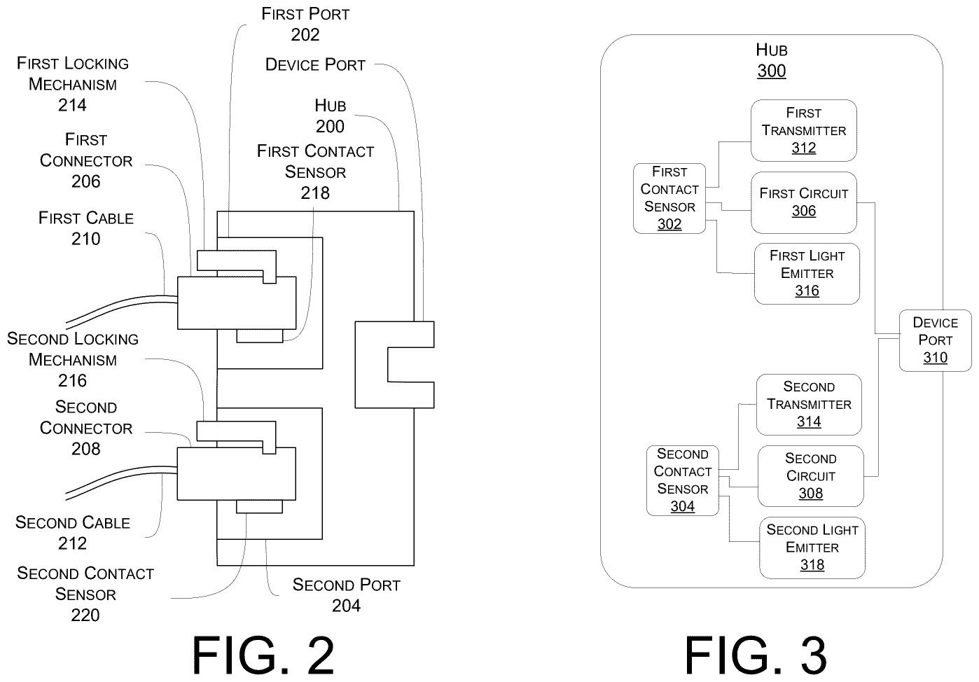

FIG. 2 illustrates an example hub 200 that includes a contact sensor and a locking mechanism. The hub 200 includes a first port 202 and a second port 204 that are configured to couple to a first connector 206 and a second connector 208 that are connected to a first cable 210 and a second cable 212, respectively.

In various implementations, a first locking mechanism 214 and a second locking mechanism 216 are configured to prevent the uncoupling of the first connector 206 and the first port 202 and the uncoupling of the second connector 208 and the second port 204, respectively. The first locking mechanism 214, in some examples, is connected to the first port 202 and the first connector 206. The second locking mechanism 216, in some examples, is connected to the second port 204 and the second connector 208.

In some examples, a first contact sensor 218 and a second contact sensor 220 are configured to detect when the first connector 206 and the second connector 208, respectively, are uncoupled from the first port 202 and the second port 204, respectively. The first contact sensor 218 and the second contact sensor 220 may include a mechanical sensor. In some examples, the first contact sensor 218 and the second contact sensor 220 are configured to detect a change in electrical signals. In some examples, the first contact sensor 218 and the second contact sensor 220 are electrically connected to a transmitter, a light emitter, a speaker, a circuit, a switch, or another component configured to transmit or output a signal indicative of the first connector 206 and the second connector 208, respectively, being uncoupled.

FIG. 3 illustrates example components of an example hub 300 as described herein. A first contact sensor 302 and a second contact sensor 304 are configured to detect when a first and second connector (e.g., a first connector 114 and a second connector 116), respectively, are uncoupled from a first and second port (e.g., a first port 128 and a second port 130), respectively.

The first contact sensor 302 and the second contact sensor 304, in some examples, are connected, by a first circuit 306 and a second circuit 308, to a device port 310. The first contact sensor 302 and the second contact sensor 304 may be a part of the first circuit 306 and the second circuit 308, respectively. The device port 310 is configured to connect to the hub 300 to a portable medical device. For instance, the device port 310 may couple to a device connector of a device cable that is connected to the portable medical device.

In various implementations, the first contact sensor 302 provides a first analog contact signal to the first circuit 306, and the second contact sensor 304 provides a second analog contact signal to the second circuit 308. The first circuit 306, in various examples, provides the first analog contact signal or a digital contact signal indicative of the first analog contact signal to the portable medical device. The second circuit 308 may provide the second analog contact signal or a second digital contact signal indicative of the second analog contact signal to the portable medical device. The portable medical device, in some examples, is configured to output an alert that the first connector and/or the second connector is uncoupled.

In some implementations the first contact sensor 302 and the second contact sensor 304 are connected to a first transmitter 312 and a second transmitter 314, respectively. The first transmitter 312 and the second transmitter 314 are configured to transmit a signal, to the portable medical device, indicating that the first connector is uncoupled from the first port or that the second connector is uncoupled from the second port, respectively. The first transmitter 312 and the second transmitter 314, in various examples, may be configured to transmit signals using one or more wireless networks. In some examples, the first transmitter 312 and the second transmitter 314 may be connected to the first contact sensor 302 and the second contact sensor 304, respectively, by a switch, a circuit, or the like. The first transmitter 312 and the second transmitter 314, in various cases, may be connected to the first circuit 306 and the second circuit 308, respectively.

In some examples, the first contact sensor 302 and the second contact sensor 304 are connected to a first light emitter 316 and a second light emitter 318, respectively. The first light emitter 316 and the second light emitter 318 may include an LED, a fluorescent light bulb, or the like. In some cases, the first light emitter 316 and the second light emitter 318 are connected to the first contact sensor 302 and the second contact sensor 304, respectively, by a switch, a circuit, or the like. The first light emitter 316 and the second light emitter 318, in some implementations, are connected to the first circuit 306 and the second circuit 308, respectively.

In various implementations, the first contact sensor 302 may be connected to at least one of the first transmitter 312, the first circuit 306, or the first light emitter 316. In some examples, the second contact sensor 304 is connected to at least one of the second transmitter 314, the second circuit 308, or the second light emitter 318.

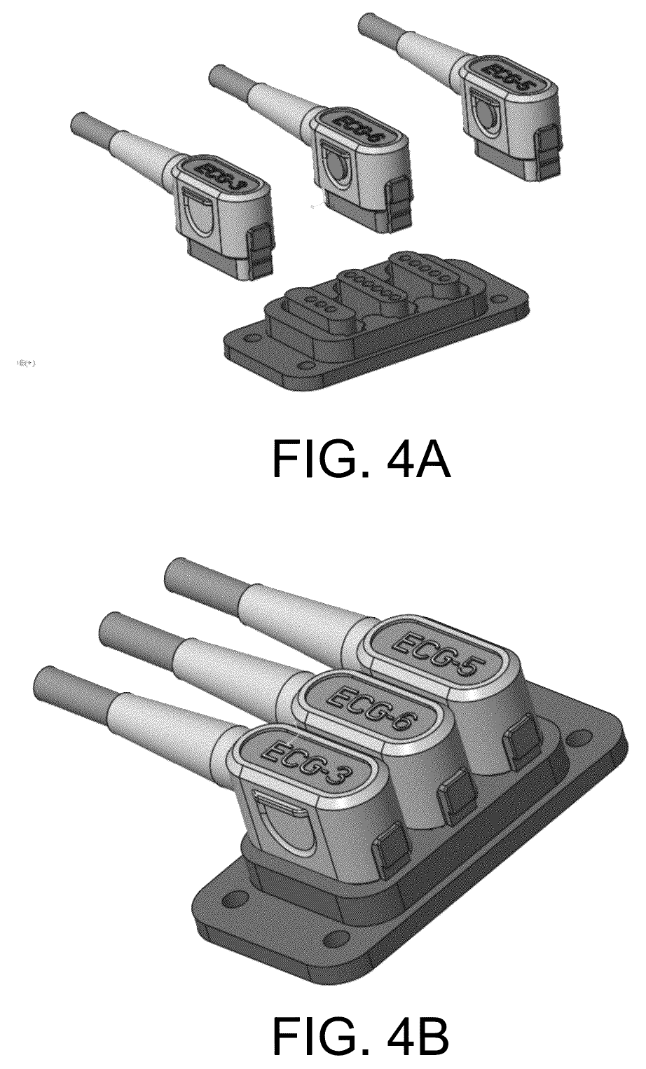

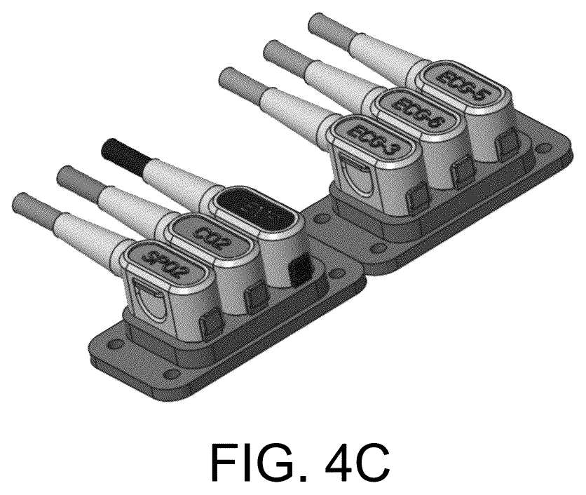

FIGS. 4A to 4C illustrate example hubs as described herein. FIGS. 4A and 4B illustrate example hubs configured to connect to sensors configured to detect different kinds of ECGs (e.g., a single-, 3-, 5-, 6-, 12-, or 15-lead ECG). For example, a hub may be configured to couple to a 3-lead ECG sensor, a 6-lead ECG sensor, and a 5-lead ECG sensor. FIG. 4C illustrates an example hub configured to connect to different kinds of sensors. For example, the hub may be configured to connect to at least one ECG sensor, a temperature sensor, a capnography sensor, and an oxygenation sensor.

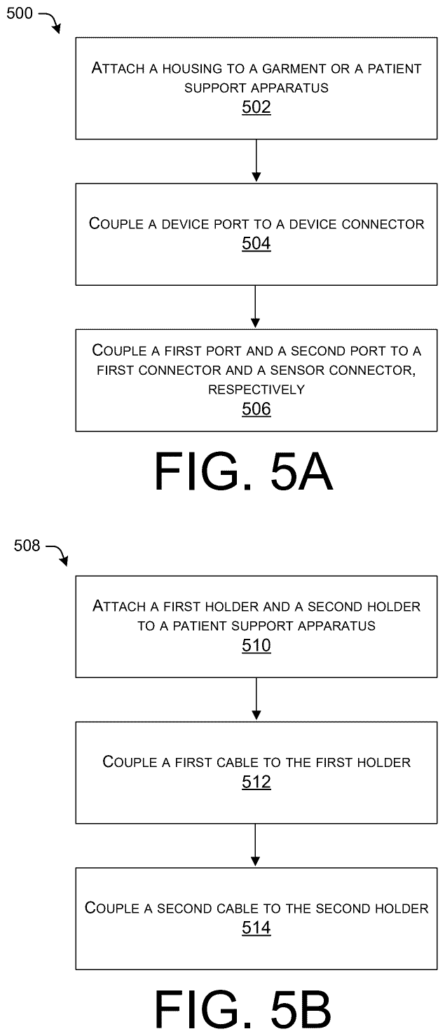

FIGS. 5A and 5B illustrates example processes 500 and 508 for connecting sensors to a portable medical device. Starting with FIG. 5A, the process 500 can be performed by an entity, which may include a user (e.g., an untrained user, a bystander, an EMT, a physician, a nurse, or the like). According to some implementations, any of the steps of process 500 may be omitted. In various implementations, the steps of process 500 may be performed in a different order than illustrated in FIG. 5A.

At 502, the entity attaches a hub to a patient support apparatus (e.g., the patient support apparatus 134) or garment. According to various implementations, the hub includes an attachment mechanism (e.g., the attachment mechanism 132) configured to attach the hub to the patient support apparatus or the garment. In some examples, the hub includes a housing, and the attachment mechanism is disposed on the housing. The attachment mechanism, in various instances, includes a hook and loop fastener, a clip, a magnet, a snap fastener, a zipper, an adhesive, or any other suitable attachment mechanism. For instance, a hook may be disposed on the housing, and a loop may be disposed on the patient support apparatus.

At 504, the entity couples a device port (e.g., the device port 122) disposed in the housing to a device connector of a device cable that is configured to connect to the portable medical device. The device port, in various examples, is a custom port, a USB port, or another type of port. The device connector, in some examples, is a custom connector, a USB connector, or another type of connector.

At 506, the entity couples a first port disposed in the housing to a first connector, and a second port disposed in the housing to a second connector. The first connector, in various instances, is connected to a first cable (e.g., the first cable 110) that is connected to a first sensor (e.g., the first accessory device 104). The second connector, in various instances, is connected to a second cable (e.g., the second cable 112) that is connected to a second sensor (e.g., the second accessory device 106). According to some examples, the first sensor and the second sensor are different. In some cases, the first port and the first connector are magnetically connected. In some cases, the second port and the second connector are magnetically connected.

Moving to FIG. 5B, the process 508 can be performed by an entity, as described above with reference to the process 500. According to some implementations, any of the steps of process 508 may be omitted. In various implementations, the steps of process 508 may be performed in a different order than illustrated in FIG. 5B.

At 510, the entity attaches a first holder (e.g., the first holder 144) and a second holder (e.g., the second holder 146) to a patient support apparatus (e.g., the patient support apparatus 134). In various examples, the first holder and the second holder are attached to the patient support apparatus by an attachment mechanism (e.g., the attachment mechanism 132). The attachment mechanism, in some cases, includes a hook and loop fastener, a clip, a magnet, a snap fastener, a zipper, or an adhesive. The patient support apparatus is, in some instances, a backboard, a stretcher, a gurney, a wheelchair, a mattress, a hospital bed, a cot, or the like. In various implementations, the attachment mechanism is configured to attach the first holder and the second holder to a surface of the patient support apparatus, a railing of the patient support apparatus, a topper disposed on the patient support apparatus, or the like.

At 512, the entity couples a first cable to the first holder. The first cable (e.g., the first cable 110) is, in some instances, attached to a first sensor (e.g., the first accessory device 104). In some examples, the first holder is configured to prevent uncoupling of the first cable from the first holder. In some examples, the first holder is configured to prevent movement of the first cable relative to the first holder. For example, a first static coefficient of friction between the first holder and the first cable may be in a range of 0.3 to 1.2. In some instances, the first static coefficient of friction may be in a range of 0.5 to 1. In various examples, the entity engages a first locking mechanism (e.g., the third locking mechanism described in reference to FIG. 1B) configured to prevent uncoupling of the first cable from the first holder and/or movement of the first cable relative to the first holder. The first locking mechanism, for instance, includes a clip, a clamp, a latch, a notch, a spring-loaded mechanism, or another mechanism configured to secure the first cable to the first holder. The first locking mechanism may be disposed on the first holder. In some examples, the first locking mechanism is configured to couple with the first holder. For instance, the entity may couple the first cable to the first holder. The entity may couple a clamp to the first holder to prevent the uncoupling of the first cable and the first holder.

At 514, the entity couples a second cable (e.g., the second cable 112) to the second holder (e.g., the second holder 146). The second cable is, in some cases, attached to a second sensor (e.g., the second accessory device 106). In various examples, the second holder is configured to prevent uncoupling of the second cable from the second holder. In some examples, the second holder is configured to prevent movement of the second cable relative to the second holder. For example, a second static coefficient of friction between the second holder and the second cable may be in a range of 0.3 to 1.2. In some instances, the second static coefficient of friction may be in a range of 0.5 to 1. In various examples, the entity engages a second locking mechanism (e.g., the fourth locking mechanism described in reference to FIG. 1B) configured to prevent uncoupling of the second cable from the second holder and/or movement of the second cable relative to the second holder. The second locking mechanism, for instance, includes a clip, a clamp, a latch, a notch, a spring-loaded mechanism, or another mechanism configured to secure the second cable to the second holder. The second locking mechanism may be disposed on the second holder. In some examples, the second locking mechanism is configured to couple with the second holder. For instance, the entity may couple the second cable to the second holder. The entity may couple a clamp to the second holder to prevent the uncoupling of the second cable and the second holder.

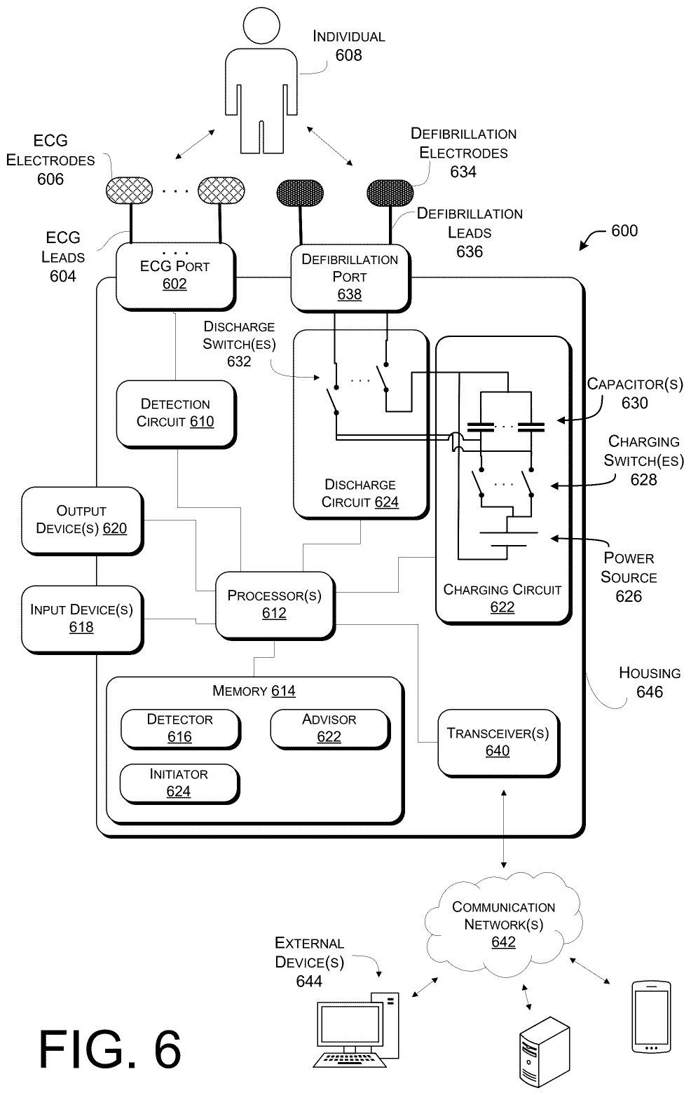

FIG. 6 illustrates an example of an external defibrillator 600 configured to perform various functions described herein. For example, the external defibrillator 600 is the portable medical device 102 described above with reference to FIGS. 1A and 1B.

The external defibrillator 600 includes a port 602 configured to connect to multiple ECG leads 604. In some cases, the ECG leads 604 are removeable from the port 602. For instance, the ECG leads 604 are plugged into the port 602. The ECG leads 604 are connected to ECG electrodes 606, respectively. In various implementations, the ECG electrodes 606 are disposed on different locations on an individual 608. A detection circuit 610 is configured to detect relative voltages between the ECG electrodes 606. These voltages are indicative of the electrical activity of the heart of the individual 608.

In various implementations of the present disclosure, the multiple ECG leads 604 are connected to the external defibrillator 600 by a hub (e.g., the first hub 118, the second hub 142). In some implementations, the port 602 may be connected to a device connector of a device cable (e.g., the device cable 126). The device cable, in some cases, is configured to connect to the hub. In some examples, the multiple electrode leads 604 are connected to the hub. In various examples, the hub is connected to one or more additional accessory device(s) (e.g., a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, a temperature sensor, or the like).

In various implementations, the ECG electrodes 606 are in contact with the different locations on the skin of the individual 608. In some examples, a first one of the ECG electrodes 606 is placed on the skin between the heart and right arm of the individual 608, a second one of the ECG electrodes 606 is placed on the skin between the heart and left arm of the individual 608, and a third one of the ECG electrodes 606 is placed on the skin between the heart and a leg (either the left leg or the right leg) of the individual 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 5-lead or 12-lead ECG signals are detected by the detection circuit 610.

The detection circuit 610 includes at least one analog circuit, at least one digital circuit, or a combination thereof. The detection circuit 610 receives the analog electrical signals from the ECG electrodes 606, via the port 602 and the ECG leads 604. The detection circuit, in various instances, receives analog electrical signal from the one or more additional sensor(s) via the port 602. In some cases, the detection circuit 610 includes one or more analog filters configured to filter noise and/or artifact from the electrical signals. The detection circuit 610 includes an analog-to-digital (ADC) in various examples. The detection circuit 610 generates a first digital signal indicative of the analog electrical signals from the ECG electrodes 606. This first digital signal can be referred to as an “ECG signal” or an “ECG.” The detection circuit 610, in some examples, generates a second digital signal indicative of the analog electrical signals from the one or more additional sensor(s). This second digital signal can be referred to as an “sensor signal.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. The impedance is generated based on the applied signal (voltage or current) and the resultant signal (current or voltage). In various cases, the impedance corresponds to respiration of the individual 608, chest compressions performed on the individual 608, and other physiological states of the individual 608. In various examples, the detection circuit 610 includes one or more analog filters configured to filter noise and/or artifact from the resultant signal. The detection circuit 610 generates a third digital signal indicative of the impedance using an ADC. This third digital signal can be referred to as an “impedance signal” or an “impedance.”

The detection circuit 610 provides the ECG signal and/or the impedance signal to one or more processors 612 in the external defibrillator 600. The detection circuit 610, in various instances, provides the sensor signal to one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores at least one of the ECG signal, the impedance signal, or the sensor signal.

In various examples, the memory 614 includes a detector 616, which causes the processor(s) 612 to determine, based on the ECG signal and/or the impedance signal, whether the individual 608 is exhibiting a particular heart rhythm. For instance, the processor(s) 612 determines whether the individual 608 is experiencing a shockable rhythm that is treatable by defibrillation. Examples of shockable rhythms include ventricular fibrillation (VF) and ventricular tachycardia (V-Tach). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal, the impedance signal, and the sensor signal.

In some examples, the memory 614 includes an advisor 623, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the individual 608. In some cases, the processor(s) 612 evaluates, based on the ECG signal, the impedance signal, or other physiological parameters, CPR being performed on the individual 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the individual 608.

The memory 614 also includes an initiator 624 which, when executed by the processor(s) 612, causes the processor(s) 612 to control other elements of the external defibrillator 600 in order to administer a defibrillation shock to the individual 608. In some examples, the processor(s) 612 executing the initiator 624 selectively causes the administration of the defibrillation shock based on determining that the individual 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal.

The processor(s) 612 is operably connected to a charging circuit 622 and a discharge circuit 625. In various implementations, the charging circuit 622 includes a power source 626, one or more charging switches 628, and one or more capacitors 630. The power source 626 includes, for instance, a battery. The processor(s) 612 initiates a defibrillation shock by causing the power source 626 to charge at least one capacitor among the capacitor(s) 630. For example, the processor(s) 612 activates at least one of the charging switch(es) 628 in the charging circuit 622 to complete a first circuit connecting the power source 626 and the capacitor to be charged. Then, the processor(s) 612 causes the discharge circuit 625 to discharge energy stored in the charged capacitor across a pair of defibrillation electrodes 634, which are in contact with the individual 608. For example, the processor(s) 612 deactivates the charging switch(es) 628 completing the first circuit between the capacitor(s) 630 and the power source 626, and activates one or more discharge switches 632 completing a second circuit connecting the charged capacitor 630 and at least a portion of the individual 608 disposed between defibrillation electrodes 634.

The energy is discharged from the defibrillation electrodes 634 in the form of a defibrillation shock. For example, the defibrillation electrodes 634 are connected to the skin of the individual 608 and located at positions on different sides of the heart of the individual 608, such that the defibrillation shock is applied across the heart of the individual 608. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 632 are controlled by the processor(s) 612, for example. In various implementations, the defibrillation electrodes 634 are connected to defibrillation leads 636. The defibrillation leads 636 are connected to a defibrillation port 638, in implementations. According to various examples, the defibrillation leads 636 are removable from the defibrillation port 638. For example, the defibrillation leads 636 are plugged into the defibrillation port 638.

In various implementations, the processor(s) 612 is operably connected to one or more transceivers 640 that transmit and/or receive data over one or more communication networks 642. For example, the transceiver(s) 640 includes a network interface card (NIC), a network adapter, a local area network (LAN) adapter, or a physical, virtual, or logical address to connect to the various external devices and/or systems. In various examples, the transceiver(s) 640 includes any sort of wireless transceivers capable of engaging in wireless communication (e.g., radio frequency (RF) communication). For example, the communication network(s) 642 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 640 includes other wireless modems, such as a modem for engaging in WI-FI®, WIGIG®, WIMAX®, BLUETOOTH®, or infrared communication over the communication network(s) 642.

The defibrillator 600 is configured to transmit and/or receive data (e.g., ECG data, impedance data, sensor data, data indicative of one or more detected heart rhythms of the individual 608, data indicative of one or more defibrillation shocks administered to the individual 608, etc.) with one or more external devices 644 via the communication network(s) 642. The external devices 644 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 642. In some examples, the external device(s) 644 is located remotely from the defibrillator 600, such as at a remote clinical environment (e.g., a hospital). According to various implementations, the processor(s) 612 causes the transceiver(s) 640 to transmit data to the external device(s) 644. In some cases, the transceiver(s) 640 receives data from the external device(s) 644 and the transceiver(s) 640 provide the received data to the processor(s) 612 for further analysis.

In various implementations, the external defibrillator 600 also includes a housing 646 that at least partially encloses other elements of the external defibrillator 600. For example, the housing 646 encloses the detection circuit 610, the processor(s) 612, the memory 614, the charging circuit 622, the transceiver(s) 640, or any combination thereof. In some cases, the input device(s) 618 and output device(s) 620 extend from an interior space at least partially surrounded by the housing 646 through a wall of the housing 646. In various examples, the housing 646 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 630, discharges the capacitor(s) 630, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, in order to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, sensor data, notifications about detected heart rhythms, and the like.

### EXAMPLE CLAUSES

Various implementations of the present disclosure are described in the following Example Clauses.

1. A hub including: a housing; a monitor port disposed in the housing and configured to be physically coupled to a monitor connector of a monitor cable that is configured to be connected to a monitor-defibrillator; an electrocardiogram (ECG) port disposed in the housing and configured to be physically coupled to an ECG connector of an ECG cable that is electrically connected to an electrode; a first circuit electrically connected to the monitor port and the ECG port; an oximetry port disposed in the housing and configured to be physically coupled to an oximetry connector of an oximetry cable that is electrically connected to a pulse oximetry sensor; a second circuit electrically connected to the monitor port and the oximetry port; and an attachment mechanism configured to attach the housing to a garment or a patient support apparatus.

2. The hub of clause 1, wherein the attachment mechanism includes a hook and loop fastener, a clip, a magnet, a snap fastener, a zipper, an adhesive, a clamp, a cable tie, a spring, a spring-loaded hose clamp, or a snap link.

3. The hub of clause 1 or 2, further including: a first contact sensor configured to detect whether the ECG connector is physically coupled to the ECG port; a first light emitter configured to emit a signal in response to the first contact sensor detecting that the ECG connector is physically uncoupled from the ECG port; a second contact sensor electrically connected to the second circuit and configured to detect whether the oximetry connector is physically coupled to the oximetry port; and a second light emitter configured to emit a second signal in response to the second contact sensor detecting that the oximetry connector is physically uncoupled from the oximetry port.

4. A hub, including: a housing; a device port disposed in the housing and configured to be physically coupled to a device connector of a device cable that is configured to be connected to a portable medical device; a first port disposed in the housing and configured to be physically coupled to a first connector of a first cable that is electrically connected to a first sensor; a first circuit connected to the device port and the first port; a second port disposed in the housing and configured to be physically coupled to a second connector of a second cable that is electrically connected to a second sensor, the second sensor being different than the first sensor; a second circuit connected to the device port and the second port; and an attachment mechanism configured to attach the housing to a garment or a patient support apparatus.

5. The hub of clause 4, wherein the device cable includes an elastic cord, a stretchable cord, or a coil cord; or wherein the garment includes a material with a Young's modulus in a range of about 0.001 to about 10.

6. The hub of clause 4 or 5, wherein the first port includes a first magnetic material and the first connector includes a second magnetic material.

7. The hub of clause 6, wherein the first magnetic material includes an electromagnet and the second magnetic material includes a rare earth magnet.

8. The hub of any of clauses 4-7, wherein the first sensor or the second sensor include an electrode, a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, or a temperature sensor.

9. The hub of any of clauses 4-8, wherein the first sensor includes an ECG sensor with a first number of electrodes and the second sensor includes an ECG sensor with a second number of electrodes, the first number and the second number being different.

10. The hub of any of clauses 4-9, further including: a first contact sensor configured to detect whether the first connector is physically coupled to the first port; a first light emitter configured to emit a first signal in response to the first contact sensor detecting that the first connector is physically uncoupled from the first port; a second contact sensor electrically connected to the second circuit and configured to detect whether the second connector is coupled to the second port; and a second light emitter configured to the second contact sensor detecting that the second connector is physically uncoupled from the second port.

11. The hub of any of clauses 4-10, further including: a first contact sensor configured to detect whether the first connector is physically coupled to the first port; a first transmitter configured to transmit a first signal to the portable medical device in response the first contact sensor detecting that the first connector is physically uncoupled from the first port; a second contact sensor electrically connected to the second circuit and configured to detect whether the second connector is coupled to the second port; and a second transmitter configured to transmit a second signal to the portable medical device in response to the second contact sensor detecting that the second connector is physically uncoupled from the second port.

12. The hub of any of clauses 4-11, wherein the attachment mechanism includes hook and loop fastener, a clip, a magnet, a snap fastener, a zipper, an adhesive, a clamp, a cable tie, a spring, a spring-loaded hose clamp, or a snap link.

13. The hub of any of clauses 4-12, wherein the attachment mechanism is configured to attach the housing to a surface of the patient support apparatus, a railing of the patient support apparatus, or a topper disposed on the patient support apparatus.

14. The hub of any of clauses 4-13, wherein the patient support apparatus includes a backboard, a stretcher, a gurney, a wheelchair, a mattress, a hospital bed, or a cot.

15. The hub of any of clauses 4-14, further including: a first locking mechanism physically connected to the first port and configured to selectively prevent uncoupling of the first connector and the first port; and a second locking mechanism physically connected to the second port and configured to selectively prevent uncoupling of the second connector and the second port.

16. The hub of clause 15, wherein the first locking mechanism or the second locking mechanism includes a magnetic connector, a twist-lock mechanism, a notch-lock mechanism, a latch, a screw-locking mechanism, or a spring-loaded mechanism.

17. The hub of any of clauses 4-16, further including: a first holder configured to be physically coupled to the first cable; and a second holder configured to be physically coupled to the second cable, wherein the first holder and the second holder are attached to the garment or the patient support apparatus.

18. A method, including: attaching a housing to a garment or a patient support apparatus; coupling a device port disposed in the housing to a device connector of a device cable that is configured to connect to a portable medical device; coupling a first port disposed in the housing to a first connector of a first cable that is electrically connected to a first sensor, the first port being connected, by a first circuit, to the device port; and coupling a second port disposed in the housing to a second connector of a second cable that is electrically connected to a second sensor, the second port being connected, by a second circuit, to the device port; wherein the second sensor is different that the first sensor.

19. The method of clause 18, wherein attaching the housing to the patient support apparatus includes: attaching the housing to a surface of the patient support apparatus, a railing of the patient support apparatus, or a topper disposed on the patient support apparatus.

20. The method of clause 18 or 19, wherein coupling the first port to the first connector includes engaging a first locking mechanism; and wherein coupling the second port to the second connector includes engaging a second locking mechanism.

21. The method of clause 20, wherein the first locking mechanism or the second locking mechanism includes a magnetic connector, a twist-lock mechanism, a notch-lock mechanism, a latch, or a screw-locking mechanism.

22. The method of any of clauses 18-21, further including: detecting, by a contact sensor configured to detect whether the first connector is physically coupled to the first port, that the first connector is uncoupled from the first port; and in response to detecting that the first connector is uncoupled from the first port, outputting an indication that the first cable is uncoupled from the first port.

23. The method of clause 22, wherein outputting the indication includes transmitting a signal to the portable medical device.

24. The method of clause 22 or 23, wherein outputting the indication includes causing a light emitter to emit a signal.

25. The method of any of clauses 18-24, further including: attaching the first cable to the garment or the patient support apparatus; and/or attaching the second cable to the garment or the patient support apparatus.

26. A hub, including: a first holder configured to be physically coupled to an ECG cable, the ECG cable being connected between a monitor-defibrillator and an electrode; a second holder configured to be physically coupled to an oximetry cable, the oximetry cable being connected between the monitor-defibrillator and a pulse oximeter; an attachment mechanism physically connected to the first holder and the second holder and configured to be removably attached to a patient support apparatus.

27. The hub of clause 26, wherein the attachment mechanism includes a hook and loop fastener, a clip, a magnet, a snap fastener, a zipper, an adhesive, a clamp, a cable tie, a spring, a spring-loaded hose clamp, or a snap link.

28. The hub of clause 26 or 27, wherein the patient support apparatus includes a backboard, a stretcher, a gurney, a wheelchair, a mattress, a hospital bed, or a cot.

29. A hub, including: a first holder configured to be removably coupled to a first cable, the first cable being connected between a portable medical device and a first sensor; a second holder configured to be removably coupled to a second cable, the second cable being connected between a portable medical device and a second sensor; an attachment mechanism physically connected to the first holder and the second holder and configured to be attached to a patient support apparatus.

30. The hub of clause 29, wherein the first holder includes a locking mechanism configured to prevent uncoupling of the first cable and the first holder.

31. The hub of clause 30, wherein the locking mechanism includes a clip, a clamp, a latch, a notch, or a spring-loaded mechanism.

32. The hub of any of clauses 29-31, wherein a first static coefficient of friction between the first holder and the first cable and a second static coefficient of friction between the second holder and the second cable are in a range of about 0.3 to about 1.2.

33. The hub of any of clauses 29-32, wherein the first holder is not adjacent to the second holder.

34. The hub of any of clauses 29-33, wherein the second sensor is different that the first sensor.

35. The hub of any of clauses 29-34, wherein the attachment mechanism includes a hook and loop fastener, a clip, a magnet, a snap fastener, a zipper, an adhesive, a clamp, a cable tie, a spring, a spring-loaded hose clamp, or a snap link.

36. The hub of any of clauses 29-35, wherein the patient support apparatus includes a backboard, a stretcher, a gurney, a wheelchair, a mattress, a hospital bed, or a cot.

37. The hub of any of clauses 29-36, wherein the first holder and the second holder are configured to be attached to a surface of the patient support apparatus, a railing of the patient support apparatus, or a topper disposed on the patient support apparatus.

38. A method, including: coupling a first cable and a first holder, the first cable being connected between a portable medical device and a first sensor; coupling a second cable and a second holder physically connected to the first holder, the second cable being connected between the portable medical device and a second sensor; attaching, by an attachment mechanism physically connected to the first holder and the second holder, the first holder and the second holder to a patient support apparatus.

39. The method of clause 38, wherein coupling the first holder to the first cable includes engaging a locking mechanism configured to prevent uncoupling of the first cable and the first holder.

40. The method of clause 39, wherein the locking mechanism includes a clip, a clamp, a latch, a notch, or a spring-loaded mechanism.

41. The method of clause 39 or 40, further including: uncoupling, by disengaging the locking mechanism, the first cable and the first holder.

42. The method of any of clauses 38-41, wherein the first holder is not adjacent to the second holder.

43. The method of any of clauses 38-42, wherein the first sensor and the second sensor are different.

44. The method of any of clauses 38-43, attaching the first holder and the second holder to the patient support apparatus includes: attaching the first holder and the second holder to a surface of the patient support apparatus, a railing of the patient support apparatus, or a topper disposed on the patient support apparatus.

45. The method of any of clauses 38-44, wherein the attachment mechanism includes a hook and loop fastener, a clip, a magnet, a snap fastener, a zipper, an adhesive, a clamp, a cable tie, a spring, a spring-loaded hose clamp, or a snap link.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A hub comprising:
a housing;
a monitor port disposed in the housing and configured to be physically coupled to a monitor connector of a monitor cable that is configured to be connected to a monitor-defibrillator;
an electrocardiogram (ECG) port disposed in the housing and configured to be physically coupled to an ECG connector of an ECG cable that is electrically connected to an electrode;
a first circuit electrically connected to the monitor port and the ECG port;
an oximetry port disposed in the housing and configured to be physically coupled to an oximetry connector of an oximetry cable that is electrically connected to a pulse oximetry sensor;
a second circuit electrically connected to the monitor port and the oximetry port; and
an attachment mechanism configured to attach the housing to a garment or a patient support apparatus.

2. The hub of claim 1, wherein the attachment mechanism comprises a hook and loop fastener, a clip, a magnet, a snap fastener, a zipper, an adhesive, a clamp, a cable tie, a spring, a spring-loaded hose clamp, or a snap link.

3. The hub of claim 1, further comprising:
a first contact sensor configured to detect whether the ECG connector is physically coupled to the ECG port;
a first light emitter configured to emit a signal in response to the first contact sensor detecting that the ECG connector is physically uncoupled from the ECG port;
a second contact sensor electrically connected to the second circuit and configured to detect whether the oximetry connector is physically coupled to the oximetry port; and
a second light emitter configured to emit a second signal in response to the second contact sensor detecting that the oximetry connector is physically uncoupled from the oximetry port.

4. A hub, comprising:
a housing;
a device port disposed in the housing and configured to be physically coupled to a device connector of a device cable that is configured to be connected to a portable medical device;
a first port disposed in the housing and configured to be physically coupled to a first connector of a first cable that is electrically connected to a first sensor;
a first circuit connected to the device port and the first port;
a second port disposed in the housing and configured to be physically coupled to a second connector of a second cable that is electrically connected to a second sensor, the second sensor being different than the first sensor;
a second circuit connected to the device port and the second port; and
an attachment mechanism configured to attach the housing to a garment or a patient support apparatus.

5. The hub of claim 4, wherein the device cable comprises an elastic cord, a stretchable cord, or a coil cord; or
wherein the garment comprises a material with a Young's modulus in a range of about 0.001 to about 10.

6. The hub of claim 4, wherein the first port comprises a first magnetic material and the first connector comprises a second magnetic material.

7. The hub of claim 6, wherein the first magnetic material comprises an electromagnet and the second magnetic material comprises a rare earth magnet.

8. The hub of claim 4, wherein the first sensor or the second sensor comprise an electrode, a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, or a temperature sensor.

9. The hub of claim 4, further comprising:
a first contact sensor configured to detect whether the first connector is physically coupled to the first port;
a first transmitter configured to transmit a first signal to the portable medical device in response the first contact sensor detecting that the first connector is physically uncoupled from the first port;
a second contact sensor electrically connected to the second circuit and configured to detect whether the second connector is coupled to the second port; and
a second transmitter configured to transmit a second signal to the portable medical device in response to the second contact sensor detecting that the second connector is physically uncoupled from the second port.

10. The hub of claim 4, wherein the attachment mechanism is configured to attach the housing to a surface of the patient support apparatus, a railing of the patient support apparatus, or a topper disposed on the patient support apparatus.

11. The hub of claim 4, further comprising:
a first locking mechanism physically connected to the first port and configured to selectively prevent uncoupling of the first connector and the first port; and
a second locking mechanism physically connected to the second port and configured to selectively prevent uncoupling of the second connector and the second port.

12. The hub of claim 11, wherein the first locking mechanism or the second locking mechanism comprises a magnetic connector, a twist-lock mechanism, a notch-lock mechanism, a latch, a screw-locking mechanism, or a spring-loaded mechanism.

13. The hub of claim 4, further comprising:
a first holder configured to be physically coupled to the first cable; and
a second holder configured to be physically coupled to the second cable,
wherein the first holder and the second holder are attached to the garment or the patient support apparatus.

14. A method, comprising:
attaching a housing to a garment or a patient support apparatus;
coupling a device port disposed in the housing to a device connector of a device cable that is configured to connect to a portable medical device;
coupling a first port disposed in the housing to a first connector of a first cable that is electrically connected to a first sensor, the first port being connected, by a first circuit, to the device port; and
coupling a second port disposed in the housing to a second connector of a second cable that is electrically connected to a second sensor, the second port being connected, by a second circuit, to the device port;
wherein the second sensor is different that the first sensor.

15. The method of claim 14, wherein attaching the housing to the patient support apparatus comprises:
attaching the housing to a surface of the patient support apparatus, a railing of the patient support apparatus, or a topper disposed on the patient support apparatus.

16. The method of claim 14, wherein coupling the first port to the first connector comprises engaging a first locking mechanism; and
wherein coupling the second port to the second connector comprises engaging a second locking mechanism.

17. The method of claim 16, wherein the first locking mechanism or the second locking mechanism comprises a magnetic connector, a twist-lock mechanism, a notch-lock mechanism, a latch, or a screw-locking mechanism.

18. The method of claim 14, further comprising:
detecting, by a contact sensor configured to detect whether the first connector is physically coupled to the first port, that the first connector is uncoupled from the first port; and
in response to detecting that the first connector is uncoupled from the first port, outputting an indication that the first cable is uncoupled from the first port.

19. The method of claim 18, wherein outputting the indication comprises:
transmitting a signal to the portable medical device; or
causing a light emitter to emit a signal.

20. The method of claim 14, further comprising:
attaching the first cable to the garment or the patient support apparatus; and/or
attaching the second cable to the garment or the patient support apparatus.
